SEQUENCE LISTING (2 sequences)

SEQ ID NO: 1 - protein, 444 aa, synthetic construct
  REGION 1..444: note = heavy chain variable region
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 219 aa, synthetic construct
  REGION 1..219: note = a light chain variable region
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0257474 A1
(43) Pub. Date: Aug. 17, 2023

(54) UVEAL MELANOMA TREATMENT USING SEA-CD40

(71) Applicant: Seagen Inc., Bothell, WA (US)

(72) Inventors: Megan O'Meara, Bothell, WA (US); Michael Schmitt, Seattle, WA (US); Amitkumar N. Mehta, Bothell, WA (US); Olivier Rixe, Bothell, WA (US)

(21) Appl. No.: 18/192,286

(22) Filed: Mar. 29, 2023

Related U.S. Application Data
(63) Continuation of application No. PCT/US2021/052688, filed on Sep. 29, 2021.
(60) Provisional application No. 63/085,765, filed on Sep. 30, 2020.

(51) Int. Cl. C07K 16/28; A61K 45/06; A61P 35/00

(52) U.S. Cl. CPC C07K 16/2878; A61K 45/06; C07K 16/2827; C07K 16/2896; A61P 35/00; A61K 2039/507

(57) ABSTRACT

This invention relates methods of using a non-fucosylated anti-CD40 antibody for treatment of uveal melanoma.

### CLAIM OF PRIORITY

Pursuant to 35 U.S.C. § 119(e), this application is a continuation of International Application No. PCT/US2021/052688, filed Sep. 29, 2021, which claims the benefit of U.S. Provisional Application No. 63/085,765, filed on Sep. 30, 2020. The entire contents of the foregoing are incorporated herein by reference.

### REFERENCE TO A SEQUENCE LISTING

This application contains a Sequence Listing that has been submitted electronically as an XML file named “49223-0061001_SL.XML.” The XML file, created on Mar. 29, 2023, is 4,155 bytes in size. The material in the XML file is hereby incorporated by reference in its entirety.

### TECHNICAL FIELD

This disclosure relates to methods of treating immunologically cold tumors including uveal melanoma using a non-fucosylated anti-CD40 antibody.

### BACKGROUND

Uveal melanoma is a malignant tumor of melanocytes in the eye. Uveal melanoma is biologically distinct from cutaneous melanoma. Uveal melanoma is the most common intraocular malignancy. The incidence of this tumor increases with age and reaches a maximum for patients in their sixties or seventies. Approximately 50% of patients die of metastases; and treatment has not improved during the last century. The average life expectancy after diagnosis of metastases is 7 months. Uveal melanoma has no standard of care. Hence, there is a high unmet need for treatment.

CD40 is a member of the tumor necrosis factor (TNF) receptor superfamily. It is a single chain type I transmembrane protein with an apparent MW of 50 kDa. CD40 is expressed by some cancer cells, e.g., lymphoma cells and several types of solid tumor cells. CD40 also functions to activate the immune system by facilitating contact-dependent reciprocal interaction between antigen-presenting cells and T cells. Although a number of anti-CD40 antibodies have been tested in clinical trials, to date none have been approved by the FDA.

### BRIEF SUMMARY

SEA-CD40, a non-fucosylated or minimally fucosylated (“non-fucosylated” and “minimally fucosylated” are used interchangeably in this application) anti-CD40 antibody, potently activates the innate immune system, and thus has the potential to treat “immunologically cold” tumors including Uveal Melanoma where a pre-existing immune reaction against the tumor is limited or absent. Additionally, combination of SEA-CD40 with a drug that inhibits an adaptive immune checkpoint (e.g. PD1/PDL1 targeting therapies such as pembrolizumab, atezolizumab, etc.) can be synergistic, as SEA-CD40 can stimulate an initial innate immune response and blockade of the PD1/PDL1 axis can allow for a sustained, adaptive immune response.

Uveal melanoma is an “immunologically cold” tumor that has been reported in a genomic study (Danaher P et al. J Immunother Cancer. 2018 Jun. 22; 6(1):63) to have the least immune infiltration of any tumors in the Cancer Genome Atlas. Thus, due to the minimal pre-existing immune response that characterizes uveal melanoma, uveal melanoma would be particularly appropriate for treatment with SEA-CD40 with or without PD1/PDL1 blockade. In some embodiments, immunologically cold tumors include uveal melanoma.

This disclosure relates to methods of treating uveal Melanoma which is not responsive to anti-PD1 or anti-PDL1 treatments. In one aspect, the disclosure relates to a method of treating uveal melanoma including administering a composition comprising an anti-CD40 antibody to a patient with uveal melanoma. The anti-CD40 antibody has 1) a heavy chain variable region comprising an amino acid sequence of SEQ ID NO:1; 2) a light chain variable region comprising an amino acid sequence of SEQ ID NO:2; and 3) a human constant region. The human constant region of the anti-40 antibody has an N-glycoside-linked sugar chain at residue N297 (EU numbering), with less than 5% of the N-glycoside-linked sugar chains at residue N297 (EU numbering) in the composition having a fucose residue. Thus the anti-CD40 antibody has minimal fucosylation. This minimally fucosylated anti-CD40 antibody is also referred to as SEA-CD40. The minimally fucosylated anti-CD40 antibody is administered at a dose of about 3 μg/kg, about 10 μg/kg, about 30 μg/kg, about 45 μg/kg, or about 60 μg/kg patient body weight.

In some embodiments, the dose of the minimally fucosylated anti-CD40 antibody is about 30 μg/kg patient body weight. In some embodiments, the minimally fucosylated anti-CD40 antibody is administered every three weeks or every six weeks. In some embodiments, the method further includes administering a chemotherapeutic agent. In some embodiments, the chemotherapeutic agent is gemcitabine, dacarbazine, temozolomide, paclitaxel, albumin-bound paclitaxel, or carboplatin. In some embodiments, the chemotherapeutic agent is gemcitabine. In some embodiments, the chemotherapeutic agent is paclitaxel. In some embodiments, the chemotherapeutic agent includes both gemcitabine and paclitaxel.

In another aspect, this disclosure relates to a method of treating uveal melanoma including administering 1) a composition comprising an anti-CD40 antibody; and 2) an anti-PD1 antibody or an anti-PDL1 antibody to a patient with uveal melanoma. The anti-CD40 antibody has 1) a heavy chain variable region comprising an amino acid sequence of SEQ ID NO:1; 2) a light chain variable region comprising an amino acid sequence of SEQ ID NO:2; and 3) a human constant region. The human constant region of the anti-CD40 antibody has an N-glycoside-linked sugar chain at residue N297 (EU numbering), with less than 5% of the N-glycoside-linked sugar chains at residue N297 (EU numbering) in the composition having a fucose residue. Thus the anti-CD40 antibody has minimal fucosylation. This minimally fucosylated anti-CD40 antibody is also referred to as SEA-CD40. The minimally fucosylated anti-CD40 antibody is administered at a dose of about 3 μg/kg, about 10 μg/kg, about 30 μg/kg, about 45 μg/kg, or about 60 μg/kg patient body weight.

In some embodiments, the anti-PD1 antibody is selected from the group consisting of Pembrolizumab, Nivolumab, Cemiplimab-rwlc, Spartalizumab, AK105, Tislelizumab, Dostarlimab, MEDI0680, Pidilizumab, AMP-224, and SHR-1210. In some embodiments, the anti-PD1 antibody is Pembrolizumab, Nivolumab, or Cemiplimab-rwlc. In some embodiments, the anti-PDL1 antibody is selected from the group consisting of Atezolizumab, Durvalumab, Avelumab, SHR-1316, MEDI4736, BMS-936559/MDX-1105, MSB0010718C, MPDL3280A, or Envafolimab. In some embodiments, the anti-PDL1 antibody is Atezolizumab, Durvalumab, or Avelumab.

In some embodiments, the dose of the minimally fucosylated anti-CD40 antibody is about 30 μg/kg pateint body weight. In some embodiments, the anti-CD40 antibody is administered every three weeks or every six weeks. In some embodiments, the method further includes administering a chemotherapeutic agent. In some embodiments, the chemotherapeutic agent is gemcitabine, dacarbazine, temozolomide, paclitaxel, albumin-bound paclitaxel, carboplatin, or paclitaxel. In some embodiments, the chemotherapeutic agent is gemcitabine. In some embodiments, the chemotherapeutic agent is paclitaxel. In some embodiments, the chemotherapeutic agent includes both gemcitabine and paclitaxel.

In some embodiments, the method of treating uveal melanoma includes administering 1) an anti-CD40 antibody and 2) an anti-PD1 antibody. In some embodiments, the anti-PD1 antibody is pembrolizumab. In some embodiments, the anti-PD1 antibody is administered at about 200 mg/kg.

In another aspect, this disclosure relates to a method of treating uveal Melanoma including administering a composition comprising an anti-CD40 antibody to a patient in need. The anti-CD40 antibody has 1) a heavy chain variable region comprising an amino acid sequence of SEQ ID NO:1; 2) a light chain variable region comprising an amino acid sequence of SEQ ID NO:2; and 3) a human constant region. The human constant region of the anti-CD40 antibody has an N-glycoside-linked sugar chain at residue N297 (EU numbering), with less than 5% of the N-glycoside-linked sugar chains at residue N297 (EU numbering) in the composition having a fucose residue. Thus the anti-CD40 antibody has minimal fucosylation. This minimally fucosylated anti-CD40 antibody is also referred to as SEA-CD40. The minimally fucosylated anti-CD40 antibody is administered at a dose of about 3 μg/kg, about 10 μg/kg, about 30 μg/kg, about 45 μg/kg, or about 60 μg/kg patient body weight. The immunologically cold tumors treated include uveal melanoma.

In some embodiments, the dose of the minimally fucosylated anti-CD40 antibody is about 30 μg/kg patient body weight. In some embodiments, the minimally fucosylated anti-CD40 antibody is administered every three weeks or every six weeks. In some embodiments, the method of treating Unveal Melanoma further includes administering a chemotherapeutic agent. In some embodiments, the chemotherapeutic agent is gemcitabine, dacarbazine, temozolomide, paclitaxel, albumin-bound paclitaxel, or carboplatin. In some embodiments, the chemotherapeutic agent is gemcitabine. In some embodiments, the chemotherapeutic agent is paclitaxel. In some embodiments, the chemotherapeutic agent includes both gemcitabine and paclitaxel.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 provides the outcome of administering SEA-CD40 and pembrolizumab to two patients with uveal melanoma. Solid lines represent Uveal Melanoma Patient 1 and Uveal Melanoma Patient 2. Dotted lines represent patients with other types of tumor.

FIGS. 2A and 2B demonstrate T cell activation in a patient with uveal melanoma after administration of SEA-CD40 and pembrolizumab. Samples were taken at pre-dose of each cycle. “C” in the x axis means cycle.

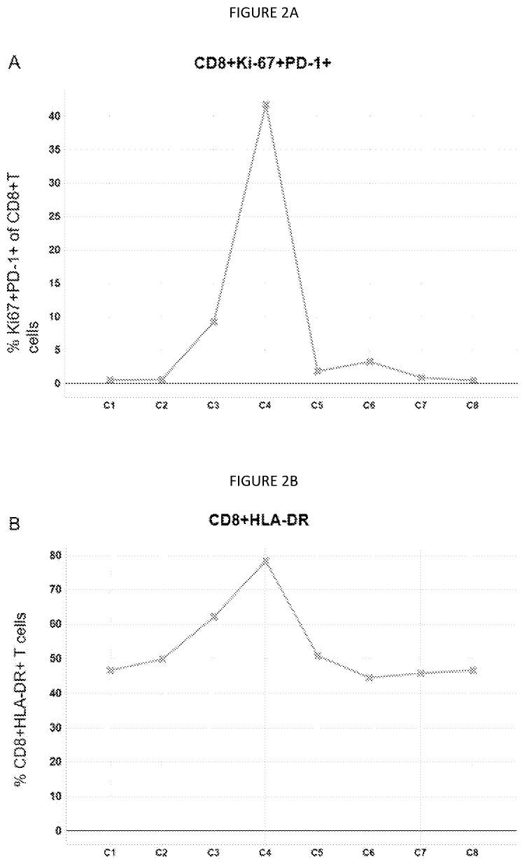

### DEFINITIONS

A “polypeptide” or “polypeptide chain” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein can also comprise non-peptidic components, such as carbohydrate groups. Carbohydrates and other non-peptidic substituents can be added to a protein by the cell in which the protein is produced, and will vary with the type of cell. Proteins are defined herein in terms of their amino acid backbone structures; substituents such as carbohydrate groups are generally not specified, but can be present nonetheless.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “antibody” is used herein to denote immunoglobulin proteins produced by the body in response to the presence of an antigen and that bind to the antigen, as well as antigen-binding fragments and engineered variants thereof. Hence, the term “antibody” includes, for example, intact monoclonal antibodies comprising full-length immunoglobulin heavy and light chains (e.g., antibodies produced using hybridoma technology) and antigen-binding antibody fragments, such as F(ab′)2 and Fab fragments. Genetically engineered intact antibodies and fragments, such as chimeric antibodies, humanized antibodies, single-chain Fv fragments, single-chain antibodies, diabodies, minibodies, linear antibodies, multivalent or multispecific (e.g., bispecific) hybrid antibodies, and the like are also included. Thus, the term “antibody” is used expansively to include any protein that comprises an antigen-binding site of an antibody and is capable of specifically binding to its antigen.

An “antigen-binding site of an antibody” is that portion of an antibody that is sufficient to bind to its antigen. The minimum such region is typically a variable domain or a genetically engineered variant thereof. Single-domain binding sites can be generated from camelid antibodies (see Muyldermans and Lauwereys, J. Mol. Recog. 12:131-140, 1999; Nguyen et al., EMBO J. 19:921-930, 2000) or from VH domains of other species to produce single-domain antibodies (“dAbs”; see Ward et al., Nature 341:544-546, 1989; U.S. Pat. No. 6,248,516 to Winter et al.). In certain variations, an antigen-binding site is a polypeptide region having only 2 complementarity determining regions (CDRs) of a naturally or non-naturally (e.g., mutagenized) occurring heavy chain variable domain or light chain variable domain, or combination thereof (see, e.g., Pessi et al., Nature 362:367-369, 1993; Qiu et al., Nature Biotechnol. 25:921-929, 2007). More commonly, an antigen-binding site of an antibody comprises both a heavy chain variable (VH) domain and a light chain variable (VL) domain that bind to a common epitope. Within the context of the present invention, an antibody can include one or more components in addition to an antigen-binding site, such as, for example, a second antigen-binding site of an antibody (which can bind to the same or a different epitope or to the same or a different antigen), a peptide linker, an immunoglobulin constant region, an immunoglobulin hinge, an amphipathic helix (see Pack and Pluckthun, Biochem. 31:1579-1584, 1992), a non-peptide linker, an oligonucleotide (see Chaudri et al., FEBS Letters 450:23-26, 1999), a cytostatic or cytotoxic drug, and the like, and can be a monomeric or multimeric protein. Examples of molecules comprising an antigen-binding site of an antibody are known in the art and include, for example, Fv, single-chain Fv (scFv), Fab, Fab′, F(ab′)₂, F(ab)_{c}, diabodies, dAbs, minibodies, nanobodies, Fab-scFv fusions, bispecific (scFv)₄-IgG, and bispecific (scFv)₂-Fab. (See, e.g., Hu et al., Cancer Res. 56:3055-3061, 1996; Atwell et al., Molecular Immunology 33:1301-1312, 1996; Carter and Merchant, Curr. Opin. Biotechnol. 8:449-454, 1997; Zuo et al., Protein Engineering 13:361-367, 2000; and Lu et al., J. Immunol. Methods 267:213-226, 2002.)

As used herein, the term “immunoglobulin” refers to a protein consisting of one or more polypeptides substantially encoded by immunoglobulin gene(s). One form of immunoglobulin constitutes the basic structural unit of native (i.e., natural) antibodies in vertebrates. This form is a tetramer and consists of two identical pairs of immunoglobulin chains, each pair having one light chain and one heavy chain. In each pair, the light and heavy chain variable regions (VL and VH) are together primarily responsible for binding to an antigen, and the constant regions are primarily responsible for the antibody effector functions. Five classes of immunoglobulin protein (IgG, IgA, IgM, IgD, and IgE) have been identified in higher vertebrates. IgG comprises the major class; it normally exists as the second most abundant protein found in plasma. In humans, IgG consists of four subclasses, designated IgG1, IgG2, IgG3, and IgG4. The heavy chain constant regions of the IgG class are identified with the Greek symbol γ. For example, immunoglobulins of the IgG1 subclass contain a γ1 heavy chain constant region. Each immunoglobulin heavy chain possesses a constant region that consists of constant region protein domains (CH1, hinge, CH2, and CH3; IgG3 also contains a CH4 domain) that are essentially invariant for a given subclass in a species. DNA sequences encoding human and non-human immunoglobulin chains are known in the art. (See, e.g., Ellison et al., DNA 1:11-18, 1981; Ellison et al., Nucleic Acids Res. 10:4071-4079, 1982; Kenten et al., Proc. Natl. Acad. Sci. USA 79:6661-6665, 1982; Seno et al., Nuc. Acids Res. 11:719-726, 1983; Riechmann et al., Nature 332:323-327, 1988; Amster et al., Nuc. Acids Res. 8:2055-2065, 1980; Rusconi and Kohler, Nature 314:330-334, 1985; Boss et al., Nuc. Acids Res. 12:3791-3806, 1984; Bothwell et al., Nature 298:380-382, 1982; van der Loo et al., Immunogenetics 42:333-341, 1995; Karlin et al., J. Mol. Evol. 22:195-208, 1985; Kindsvogel et al., DNA 1:335-343, 1982; Breiner et al., Gene 18:165-174, 1982; Kondo et al., Eur. J. Immunol. 23:245-249, 1993; and GenBank Accession No. J00228.) For a review of immunoglobulin structure and function, see Putnam, The Plasma Proteins, Vol V, Academic Press, Inc., 49-140, 1987; and Padlan, Mol. Immunol. 31:169-217, 1994. The term “immunoglobulin” is used herein for its common meaning, denoting an intact antibody, its component chains, or fragments of chains, depending on the context.

Full-length immunoglobulin “light chains” (about 25 Kd or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 Kd or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin light or heavy chain variable region (also referred to herein as a “light chain variable domain” (“VL domain”) or “heavy chain variable domain” (“VH domain”), respectively) consists of a “framework” region interrupted by three hypervariable regions, also called “complementarity determining regions” or “CDRs.” The framework regions serve to align the CDRs for specific binding to an epitope of an antigen. Thus, the term “hypervariable region” or “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917, 1987; Chothia et al., Nature 342:878-883, 1989. Kabat also provides a widely used numbering convention (Kabat numbering) in which corresponding residues between different heavy chains or between different light chains are assigned the same number. CDRs 1, 2, and 3 of a VL domain are also referred to herein, respectively, as CDR-L1, CDR-L2, and CDR-L3; CDRs 1, 2, and 3 of a VH domain are also referred to herein, respectively, as CDR-H1, CDR-H2, and CDR-H3.

Unless the context dictates otherwise, the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

The term “chimeric antibody” refers to an antibody having variable domains derived from a first species and constant regions derived from a second species. Chimeric immunoglobulins or antibodies can be constructed, for example by genetic engineering, from immunoglobulin gene segments belonging to different species. The term “humanized antibody,” as defined infra, is not intended to encompass chimeric antibodies. Although humanized antibodies are chimeric in their construction (i.e., comprise regions from more than one species of protein), they include additional features (i.e., variable regions comprising donor CDR residues and acceptor framework residues) not found in chimeric immunoglobulins or antibodies, as defined herein.

The term “humanized VH domain” or “humanized VL domain” refers to an immunoglobulin VH or VL domain comprising some or all CDRs entirely or substantially from a non-human donor immunoglobulin (e.g., a mouse or rat) and variable region framework sequences entirely or substantially from human immunoglobulin sequences. The non-human immunoglobulin providing the CDRs is called the “donor” and the human immunoglobulin providing the framework is called the “acceptor.” In some instances, humanized antibodies can retain non-human residues within the human variable domain framework regions to enhance proper binding characteristics (e.g., mutations in the frameworks can be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

Specific binding of an antibody to its target antigen means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not, however, necessarily imply that a monoclonal antibody binds one and only one target.

With regard to proteins as described herein, reference to amino acid residues corresponding to those specified by SEQ ID NO includes post-translational modifications of such residues.

The term “diluent” as used herein refers to a solution suitable for altering or achieving an exemplary or appropriate concentration or concentrations as described herein.

The term “container” refers to something into which an object or liquid can be placed or contained, e.g., for storage (for example, a holder, receptacle, vessel, or the like).

The term “administration route” includes art-recognized administration routes for delivering a therapeutic protein such as, for example, parenterally, intravenously, intramuscularly, or subcutaneously. For administration of an antibody for the treatment of cancer, administration into the systemic circulation by intravenous or subcutaneous administration can be desired. For treatment of a cancer characterized by a solid tumor, administration can also be localized directly into the tumor, if so desired.

The term “treatment” refers to the administration of a therapeutic agent to a patient, who has a disease with the purpose to cure, heal, alleviate, delay, relieve, alter, remedy, ameliorate, improve or affect the disease.

The term “patient” includes human and other mammalian subjects that receive either prophylactic or therapeutic treatment.

The term “effective amount,” “effective dose,” or “effective dosage” refers to an amount that is sufficient to achieve or at least partially achieve the desired effect, e.g., sufficient to inhibit the occurrence or ameliorate one or more symptoms of a disease or disorder. An effective amount of a pharmaceutical composition is administered in an “effective regime.” The term “effective regime” refers to a combination of amount of the composition being administered and dosage frequency adequate to accomplish prophylactic or therapeutic treatment of the disease or disorder.

As used herein, the term “about” denotes an approximate range of plus or minus 10% from a specified value. For instance, the language “about 20 μg/kg” encompasses a range of 18-22 μg/kg. As used herein, about also includes the exact amount. Hence “about 20 μg/kg” means “about 20 μg/kg” and also “20 ag/kg.”

### DETAILED DESCRIPTION

### Uveal Melanoma is an Immunologically Cold Tumor

Uveal melanoma has been reported to exhibit the least immune activity among all tumors analyzed in the Cancer Genome Atlas (Danaher P et al. J Immunother Cancer. 2018 Jun. 22; 6(1):63), and can thus be considered an “immunologically cold” tumor. Uveal melanoma is the most common primary adult malignancy of the eye with more than two thousand new cases and more than two hundred deaths in the United States annually. Uveal melanoma has limited treatment options. Current NCCN treatment guidelines recommend consideration of a clinical trial as the first line of treatment for patients with metastatic uveal melanoma, because in general, there are no systemic therapies that have reliably improved overall survival in this setting (J Natl Compr Canc Netw. 2018 May; 16(5S):646-650. doi: 10.6004/jnccn.2018.0042).

Immune checkpoint inhibitors such as PD1 and PDL1 antibodies rarely confer durable remissions in patients with metastatic uveal melanoma (Algazi et al. Cancer. 2016 Nov. 15; 122(21): 3344-335). Approved therapies that target immune cell checkpoints such as the PD1/PDL1 axis are directed toward reinvigorating a pre-existing immune response. However, in many cases, the immune system fails to mount a meaningful response against a tumor. As a consequence, therapies targeting adaptive immune checkpoints are often ineffective. For example, even in cancers that are stereotypically classified as “immunologically hot” such as cutaneous melanoma, PD1/PDL1 targeting therapies can be ineffective in the majority of patients, with an objective response rate for pembrolizumab in therapy-naïve cutaneous melanoma patients of 42% (Long G V et al, J Clin Oncol 2018; 36S:ASCO #9503). In other “immunologically cold” tumors such as uveal melanoma, PD1/PDL1 targeting therapies have little or no activity, with an objective response rate of such agents in metastatic uveal melanoma of less than 4% (Algazi et al. Cancer. 2016 Nov. 15; 122(21): 3344-3353). In some embodiments, immunologically cold tumors include uveal melanoma. As a consequence, there is a substantial need for therapies that can initiate an immune response independent of PD1/PDL1 blockade.

This disclosure demonstrates that CD40 targeting antibodies, alone or in combination with other therapeutic agent, can be effective treatment for uveal Melanoma. Activation of CD40 with a CD40 targeting antibody can initiate an innate immune reaction against a tumor. In addition, CD40 antibody afucosylation via SEA technology can result in greatly enhanced potency relative to a conventional CD40 antibody, therefore resulting in further innate immune stimulation. For the first time, CD40 agonism (particularly with a non-fucosylated antibody) is shown to trigger a de novo immune response to uveal melanoma in a patient.

Combination of a CD40 antibody with a drug resulting in PD1/PDL1 blockade (e.g. pembrolizumab) can also result in a sustained immune response, via innate immune activation from CD40 binding and sustained activation of the adaptive immune system from PD1/PDL1 blockade. This approach of simultaneously “pressing the gas” of the immune system via CD40 agonism, in conjunction with “releasing the brakes” via PD1/PDL1 blockade, can be particularly effective in “immunologically cold” tumors including uveal melanoma.

This disclosure provides description of the activity of a non-fucosylated anti-CD40 antibody, SEA-CD40, in the treatment of uveal melanoma, an “immunologically cold” tumor. SEA-CD40 is an agonistic antibody and has enhanced binding to Fc7 receptors III and, exhibits enhanced activation of the CD40 signaling pathway. Because of its enhanced activation of the CD40 pathway SEA-CD40 is a potent activator of the immune system. The enhanced activation of the immune system allows SEA-CD40 to be dosed at low levels, as compared to a fucosylated parent antibody.

### CD40

CD40 is a member of the tumor necrosis factor (TNF) receptor superfamily. It is a single chain type I transmembrane protein with an apparent MW of 50 kDa. Its mature polypeptide core consists of 237 amino acids, of which 173 amino acids comprise an extracellular domain (ECD) organized into 4 cysteine-rich repeats that are characteristic of TNF receptor family members. Two potential N-linked glycosylation sites are present in the membrane proximal region of the ECD, while potential O-linked glycosylation sites are absent. A 22 amino acid transmembrane domain connects the ECD with the 42 amino acid cytoplasmic tail of CD40. Sequence motifs involved in CD40-mediated signal transduction have been identified in the CD40 cytoplasmic tail. These motifs interact with cytoplasmic factors called TNF-R-associated factors (TRAFs) to trigger multiple downstream events including activation of MAP kinases and NFκB, which in turn modulate the transcriptional activities of a variety of inflammation-, survival-, and growth-related genes. See, e.g., van Kooten and Banchereau, J. Leukoc. Biol. 67:2-17 (2000); Elgueta et al., Immunol. Rev. 229:152-172 (2009).

Within the hematopoietic system, CD40 can be found on B cells at multiple stages of differentiation, monocytes, macrophages, platelets, follicular dendritic cells, dendritic cells (DC), eosinophils, and activated T cells. In normal non-hematopoietic tissues, CD40 has been detected on renal epithelial cells, keratinocytes, fibroblasts of synovial membrane and dermal origins, and activated endothelium. A soluble version of CD40 is released from CD40-expressing cells, possibly through differential splicing of the primary transcript or limited proteolysis by the metalloproteinase TNFα converting enzyme. Shed CD40 can potentially modify immune responses by interfering with the CD40/CD40L interaction. See, e.g., van Kooten and Banchereau, J. Leukoc. Biol. 67:2-17 (2000); Elgueta et al., Immunol. Rev. 229:152-172 (2009).

The endogenous ligand for CD40 (CD40L) is a type II membrane glycoprotein of 39 kDa also known as CD154. CD40L is a member of the TNF superfamily and is expressed as a trimer on the cell surface. CD40L is transiently expressed on activated CD4+, CD8+, and T6 T cells. CD40L is also detected at variable levels on purified monocytes, activated B cells, epithelial and vascular endothelial cells, smooth muscle cells, and DCs, but the functional relevance of CD40L expression on these cell types has not been clearly defined (van Kooten 2000; Elgueta 2009). However, expression of CD40L on activated platelets has been implicated in the pathogenesis of thrombotic diseases. See, e.g., Ferroni et al., Curr. Med. Chem. 14:2170-2180 (2007).

The best-characterized function of the CD40/CD40L interaction is its role in contact-dependent reciprocal interaction between antigen-presenting cells and T cells. See, e.g., van Kooten and Banchereau, J. Leukoc. Biol. 67:2-17 (2000); Elgueta et al., Immunol. Rev. 229:152-172 (2009). Binding of CD40L on activated T cells to CD40 on antigen-activated B cells not only drives rapid B cell expansion, but also provides an essential signal for B cells to differentiate into either memory B cells or plasma cells. CD40 signaling is responsible for the formation of germinal centers in which B cells undergo affinity maturation and isotype switching to acquire the ability to produce high affinity antibodies of the IgG, IgA, and IgE isotypes. See, e.g., Kehry, J. Immunol. 156:2345-2348 (1996). Thus, individuals with mutations in the CD40L locus that prevent functional CD40/CD40L interaction suffer from the primary immunodeficiency X-linked hyper-IgM syndrome that is characterized by over-representation of circulating IgM and the inability to produce IgG, IgA, and IgE. These patients demonstrate suppressed secondary humoral immune responses, increased susceptibility to recurrent pyrogenic infections, and a higher frequency of carcinomas and lymphomas. Gene knockout experiments in mice to inactivate either CD40 or CD40L locus reproduce the major defects seen in X-linked hyper-IgM patients. These KO mice also show impaired antigen-specific T cell priming, suggesting that the CD40L/CD40 interaction is also a critical factor for mounting cell-mediated immune responses. See, e.g., Elgueta et al., Immunol. Rev. 229:152-172 (2009).

The immune-stimulatory effects of CD40 ligation by CD40L or anti-CD40 in vivo have correlated with immune responses against syngeneic tumors. See, e.g., French et al., Nat. Med. 5:548-553 (1999). A deficient immune response against tumor cells can result from a combination of factors such as expression of immune checkpoint molecules, such as PD1 or CTLA-4, decreased expression of MHC antigens, poor expression of tumor-associated antigens, appropriate adhesion, or co-stimulatory molecules, and the production of immunosuppressive proteins like TGFβ by the tumor cells. CD40 ligation on antigen presenting and transformed cells results in up-regulation of adhesion proteins (e.g., CD54), co-stimulatory molecules (e.g., CD86) and MHC antigens, as well as inflammatory cytokine secretion, thereby potentially inducing and/or enhancing the antitumor immune response, as well as the immunogenicity of the tumor cells. See, e.g., Gajewski et al., Nat. Immunol. 14:1014-1022 (2013).

A primary consequence of CD40 cross-linking is DC activation (often termed licensing) and potentiation of myeloid and B cells ability to process and present tumor-associated antigens to T cells. Besides having a direct ability to activate the innate immune response, a unique consequence of CD40 signaling is APC presentation of tumor-derived antigens to CD8+ cytotoxic T cell (CTL) precursors in a process known as ‘cross-priming’. This CD40-dependent activation and differentiation of CTL precursors by mature DCs into tumor-specific effector CTLs can enhance cell-mediated immune responses against tumor cells. See, e.g., Kurts et al., Nat. Rev. Immunol. 10:403-414 (2010).

Agonistic CD40 mAbs including dacetuzumab, the SEA-CD40 parent molecule, have shown encouraging clinical activity in single-agent and combination chemotherapy settings. Dacetuzumab demonstrated some clinical activity in a phase 1 study in NHL and a phase 2 study in diffuse large B-cell lymphoma (DLBCL). See, e.g., Advani et al., J. Clin. Oncol. 27:4371-4377 (2009) and De Vos et al., J. Hematol. Oncol. 7:1-9 (2014). Additionally CP-870,893, a humanized IgG2 agonist antibody to CD40, showed encouraging activity in solid tumor indications when combined with paclitaxel or carboplatin or gemcitabine. In these studies, activation of antigen presenting cells, cytokine production, and generation of antigen-specific T cells were seen. See, e.g., Beatty et al., Clin. Cancer Res. 19:6286-6295 (2013) and Vonderheide et al., Oncoimmunology 2:e23033 (2013)

### Anti-CD40 Antibodies

Anti-CD40 antibodies, e.g., S2C6, have been disclosed in US20170333556A1, which is herein incorporated by reference. The S2C6 antibody is a partial agonist of the CD40 signaling pathway and thus has the following activities: binding to human CD40 protein, binding to cynomolgus CD40 protein, activation of the CD40 signaling pathway, potentiation of the interaction of CD40 with its ligand, CD40L. See, e.g., U.S. Pat. No. 6,946,129, which is herein incorporated by reference.

Humanized anti-CD40 antibodies, e.g., humanized S2C6 (hS2C6), have been disclosed in U.S. Pat. No. 8,303,955B2 and U.S. Pat. No. 8,492,531B2, both of which are herein incorporated by reference.

Non-fucosylated anti-CD40 antibodies, e.g. nf hS2C6 or SEA-CD40 have been disclosed in US20170333556A1. In addition to enhanced binding to Fc receptors, SEA-CD40 also enhances activity of the CD40 pathway, as compared to the parent antibody, dacetuzumab. The SEA-CD40 antibody thus, is administered to patients at lower doses and using different schedules of administration.

SEA-CD40 exhibits enhanced binding to FcγIII receptors, and enhanced ability to activate the CD40 signaling pathway in immune cells, as described in US20170333556A1. Methods of making the non-fucosylated antibodies including SEA-CD40 are also disclosed in US20170333556A1.

### Dosage and Administration of SEA-CD40 for Treating Uveal Melanoma

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

SEA-CD40 is administered intravenously. In other embodiments, SEA-CD40 is administered subcutaneously. In a further embodiment, SEA-CD40 is administered subcutaneously at the site of a tumor.

As an example, SEA-CD40 can be adminstered to uveal melanoma patients at levels between about 0.1 to about 70 μg/kg (ag antibody per kilogram patient body weight). Other possible dosage ranges include about 1 μg/kg to about 60 μg/kg, about 10 μg/kg to about 50 μg/kg, and about 20 μg/kg to about 40 μg/kg. Other possible dosage ranges include the following: about 1 μg/kg to about 5 μg/kg, about 5 μg/kg to about 10 μg/kg, about 10 μg/kg to about 15 μg/kg, about 15 μg/kg to about 20 μg/kg, about 20 μg/kg to about 25 μg/kg, about 25 μg/kg to about 30 μg/kg, about 30 μg/kg to about 35 μg/kg, about 35 μg/kg to about 40 μg/kg, about 40 μg/kg to about 45 μg/kg, about 45 μg/kg to about 50 μg/kg, about 50 ag/kg to about 55 μg/kg, and about 55 μg/kg to about 60 μg/kg.

In other embodiments, SEA-CD40 is administered to uveal melanoma patients at about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 11 μg/kg, about 12 μg/kg, about 13 μg/kg, about 14 μg/kg, about 15 μg/kg, about 16 μg/kg, about 17 μg/kg, about 18 μg/kg, about 19 μg/kg, about 20 μg/kg, about 21 μg/kg, about 22 μg/kg, about 23 μg/kg, about 24 μg/kg, about 25 μg/kg, about 26 μg/kg, about 27 μg/kg, about 28 μg/kg, about 29 μg/kg, about 30 μg/kg, about 31 μg/kg, about 32 μg/kg, about 33 μg/kg, about 34 μg/kg, about 35 μg/kg, about 36 μg/kg, about 37 μg/kg, about 38 μg/kg, about 39 μg/kg, about 40 μg/kg, about 41 μg/kg, about 42 μg/kg, about 43 μg/kg, about 44 μg/kg, about 45 μg/kg, about 46 μg/kg, about 47 μg/kg, about 48 μg/kg, about 49 μg/kg, about 50 μg/kg, about 51 μg/kg, about 52 μg/kg, about 53 μg/kg, about 54 μg/kg, about 55 μg/kg, about 56 μg/kg, about 57 μg/kg, about 58 μg/kg, about 59 μg/kg, about 60 μg/kg, about 61 μg/kg, about 62 μg/kg, about 63 μg/kg, about 64 μg/kg, about 65 μg/kg, about 66 μg/kg, about 67 μg/kg, about 68 μg/kg, about 69 μg/kg, or about 70 μg/kg. In preferred embodiments, SEA-CD40 is administered to uveal melanoma patients at about 3 μg/kg, about 10 μg/kg, about 30 μg/kg, about 45 μg/kg, or about 60 μg/kg. In a more preferred embodiment, SEA-CD40 is administered to uveal melanoma patients at about 30 μg/kg.

SEA-CD40 can be administered at different intervals including one week intervals, two weeks intervals, three week intervals, four weeks interval, five week intervals, six week intervals, seven week intervals, eight week intervals, nine week intervals, ten week intervals, eleven week intervals, twelve week intervals, thirteen week intervals, or fourteen week intervals. In some embodiments, intervals are on a monthly schedule, e.g., one month intervals, two month intervals, or three month intervals. In some embodiments, intervals are based on cycles wherein each cycle can comprise one or more administrations of SEA-CD40. Exemplary lengths of each cycle include one week, two weeks, three weeks, four weeks, five week, six weeks, seven weeks, eight weeks, nine weeks, ten weeks, eleven weeks, and twelve weeks. The lengths of cycles can be different from one cycle to the next. SEA-CD40 can be administered on any one or more days in each cycle. In some embodiments, SEA-CD40 is administered on the first day of a cycle. In some embodiments, SEA-CD40 is administered on the first day of a cycle of three weeks in a treatment period of one cycle, two cycles, three cycles, four cycles, five cycles, six cycles, seven cycles, eight cycles, nine cycles, ten cycles, eleven cycles, twelve cycles, thirteen cycles, fourteen cycles, fifteen cycles, or sixteen cycles.

### SEA-CD40 Combination Therapy for Treating Uveal Melanoma

SEA-CD40 can be used in combination with other therapeutic agents for treating uveal melanoma.

In some embodiments, SEA-CD40 can be administered in combination with an anti-PD1 antibody to treat uveal melanoma. In some embodiments, SEA-CD40 is administered in combination with Pembrolizumab, Nivolumab, Cemiplimab-rwlc, Spartalizumab, AK105, Tislelizumab, Dostarlimab, MEDI0680, Pidilizumab, AMP-224, or SHR-1210. In some embodiments, SEA-CD40 is administered with one or more of Pembrolizumab, Nivolumab, and Cemiplimab-rwlc. In some embodiments, SEA-CD40 is administered in combination with pembrolizumab. In some embodiments, SEA-CD40 is administered in combination with Nivolumab. In some embodiments, SEA-CD40 is administered in combination with Cemiplimab-rwlc.

In some embodiments, SEA-CD40 can be administered in combination with an anti-PDL1 antibody to treat uveal melanoma. In some embodiments, SEA-CD40 is administered in combination with Atezolizumab, Durvalumab, Avelumab, SHR-1316, MEDI4736, BMS-936559/MDX-1105, MSB0010718C, MPDL3280A, or Envafolimab. In some embodiments, SEA-CD40 is administered with one or more of Atezolizumab, Durvalumab, Avelumab. In some embodiments, SEA-CD40 is administered in combination with Atezolizumab. In some embodiments, SEA-CD40 is administered in combination with Durvalumab. In some embodiments, SEA-CD40 is administered in combination with Avelumab.

In some embodiments, SEA-CD40 can be administered in combination with an anti-CTLA-4 antibody to treat uveal melanoma. In some embodiments, SEA-CD40 is administered in combination with ipilimumab or tremelimumab to treat uveal melanoma.

In some embodiments, SEA-CD40 can be administered in combination with other antibodies that block the function of immune checkpoint proteins include antibodies directed against, e.g., LAG3, CD47 and TIM3. In some embodiments, SEA-CD40 is administered in combination with REGN3767, Relatlimab, LAG-52, MK-4280, or GSK2831781.

In some embodiments, SEA-CD40 can be administered in combination with antibodies against 41BB, CD27, ICOS, or OX40. Anti-41BB antibodies include, e.g., Urelumab and Utomilumab. Anti-OX40 antibodies include, e.g., INCAGN1949, MEDI6469 and MEDI6383.

SEA-CD40 can be used in combination with chemotherapeutic agents or chemotherapy to treat uveal melanoma. In most humans, millions of cells die via apoptosis and are removed without generating an immune response. However, after treatment with some chemotherapeutic agents, immune cells have been observed to infiltrate tumors. Thus, some tumor cells killed by chemotherapeutic agents act as vaccines and raise a tumor-specific immune response. This phenomenon is referred to as immunogenic cell death (ICD). See, e.g., Kroemer et al., Annu. Rev. Immunol., 31:51-72 (2013). The ability of a chemotherapeutic agent to induce ICD can be determined experimentally. Two criteria must be met. First, injection of an immunocompetent mouse with cancer cells that have been treated in vitro with a chemotherapeutic agent must elicit a protective immune response that is specific for tumor antigens, in the absence of adjuvant. Second, ICD occurring in vivo, e.g., a mouse syngeneic model with treatment using a potential ICD-inducing chemotherapeutic agent, must drive an immune response in the tumor that is dependent on the immune system.

Chemotherapeutic agents that induce ICD include, e.g., anthracyclines, anti-EGFR antibodies, bortezomib, cyclophosphamide, gemcitabine, irradiation of the tumor, and oxaliplatin. SEA-CD40 can be used in combination with any of these agents to generate an enhanced immune response and treat uveal melanoma in a patient. In some embodiments, SEA-CD40 is used in combination with gemcitabine, dacarbazine, temozolomide, paclitaxel, albumin-bound paclitaxel, or carboplatin.

In some embodiments, SEA-CD40 is used in combination with two or more agents to treat uveal melanoma. In some embodiments, SEA-CD40 is used in combination with a chemotherapeutic agent and an immune checkpoint inhibitor. In some embodiments, SEA-CD40 is used in combination with a chemotherapeutic agent and an anti-PD1 antibody. In some embodiments, SEA-CD40 is used in combination with a chemotherapeutic agent and an anti-PD1 antibody selected from Pembrolizumab, Nivolumab, Cemiplimab-rwlc, Spartalizumab, AK105, Tislelizumab, Dostarlimab, MEDI0680, Pidilizumab, AMP-224, and SHR-1210. In some embodiments, SEA-CD40 is used in combination with a chemotherapeutic agent and one or more of Pembrolizumab, Nivolumab, and Cemiplimab-rwlc. In some embodiments, SEA-CD40 is used in combination with a chemotherapeutic agent and Pembrolizumab. In some embodiments, SEA-CD40 is used in combination with a chemotherapeutic agent and Nivolumab. In some embodiments, SEA-CD40 is used in combination with a chemotherapeutic agent and Cemiplimab-rwlc. In any of the above-mentioned embodiments, the chemotherapeutic agent used in combination can be gemcitabine, dacarbazine, temozolomide, paclitaxel, albumin-bound paclitaxel, carboplatin, or paclitaxel. In some embodiments, the chemotherapeutic agent used in any of the above-mentioned combinations is gemcitabine. In some embodiments, the chemotherapeutic agent used in any of the above-mentioned combinations is paclitaxel. In some embodiments, the chemotherapeutic agent used in any of the above-mentioned combinations includes both gemcitabine and paclitaxel.

In some other embodiments, SEA-CD40 is used in combination with a chemotherapeutic agent and an anti-PDL1 antibody. In some embodiments, SEA-CD40 is used in combination with a chemotherapeutic agent and an anti-PDL1 antibody selected from Atezolizumab, Durvalumab, Avelumab, SHR-1316, MEDI4736, BMS-936559/MDX-1105, MSB0010718C, MPDL3280A, and Envafolimab. In some embodiments, SEA-CD40 is used in combination with a chemotherapeutic agent and one or more of Atezolizumab, Durvalumab, and Avelumab. In some embodiments, SEA-CD40 is used in combination with a chemotherapeutic agent and Atezolizumab. In some embodiments, SEA-CD40 is used in combination with a chemotherapeutic agent and Durvalumab. In some embodiments, SEA-CD40 is used in combination with a chemotherapeutic agent and Avelumab. In any of the above-mentioned embodiments, the chemotherapeutic agent used in combination can be gemcitabine, dacarbazine, temozolomide, paclitaxel, albumin-bound paclitaxel, carboplatin, or paclitaxel. In some embodiments, the chemotherapeutic agent used in any of the above-mentioned combinations is gemcitabine. In some embodiments, the chemotherapeutic agent used in any of the above-mentioned combinations is paclitaxel. In some embodiments, the chemotherapeutic agent used in any of the above-mentioned combinations includes both gemcitabine and paclitaxel.

Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications can be practiced within the scope of the appended claims.

### EXAMPLES

The following examples are offered to illustrate, but not to limit the claimed invention.

### Example 1: Treating Uveal Melanoma with SEA-CD40

In a phase I clinical trial, two patients with uveal melanoma (patient 1 and patient 2) were treated with SEA-CD40 and pembrolizumab.

The patients remained on-study for 4.6 months and 6.3 months prior to progression. These are considered clinically meaningful responses for uveal melanoma, given that pembrolizumab monotherapy results in 6 month disease control in only 9% of patients (Algazi et al. Cancer. 2016 Nov. 15; 122(21): 3344-335). Further, both patients obtained a response of stable disease after SEA-C40+pembrolizumab treatment, despite having a best response of progressive disease to two or more prior lines of immunotherapy. In addition, SEA-CD40 and pembrolizumab resulted in the longest duration of disease control of any of the immunotherapies received by either patient.

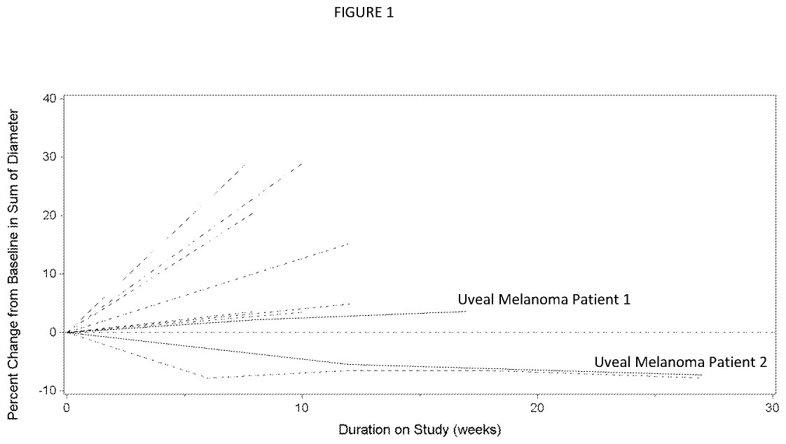

Prior to treatment of SEA-CD40 in combination of pembrolizumab, patient 1 had a baseline disease of 3.6 cm aortic lymph node (LN), 2.1 cm right upper lobe of lung (RUL), 4.0 cm liver, 4.3 cm portal LN, b/l hilar nodes, multiple pulmonary+liver nodes, and other LNs. FIG. 1 demonstrates stable disease of patient 1 when treated with SEA-40 and pembrolizumab.

Patient 1 was on treatment regimen of SEA-CD40 (10 μg/kg) in combination with pembrolizumab (200 mg/kg) for a period of 140 days (20 weeks, 7 cycles) during which stable disease was observed up to 18 weeks (6 cycles). For each cycle (3 weeks), SEA-CD40 was given intravenously on Day 1, and pembrolizumab was given intravenously on Day 2.

Prior to treatment of SEA-CD40 in combination of pembrolizumab, patient 2 had a baseline disease of 2.0 cm RUL, 1.8 cm LUL, 3.7 cm liver, 3.5 cm liver, and peritoneal carcinomatosis. Patient 2 was previously administered several other treatment regimens. Stable disease was not observed during any of the previous treatment regimens. The failed treatment regimens include 1) pembrolizumab in combination with indoximod; 2) ipilimumab in combination with indoximod; and 3) Y90 chemoembolization. Each of the previous treatment regimens was stopped due to progressive disease (not stabilized by treatment). FIG. 1 demonstrates stable disease of patient 2 when treated with SEA-40 and pembrolizumab.

Patient 2 was on treatment regimen of SEA-CD40 (3 μg/kg) in combination with pembrolizumab (200 mg/kg) for a period of 191 days during which stable disease was observed up to 27 weeks (9 cycles). For each cycle (3 weeks), SEA-CD40 was given intravenously on Day 1, and pembrolizumab was given intravenously on Day 2.

More patients are treated with SEA-CD40 at about 3 μg/kg for multiple cycles of administration, each cycle being 3 weeks. SEA-CD40 is administered one day of each cycle, e.g. day one of each cycle; or on two or more days in each cycle. Patients exhibit stable disease without progression for a medically significant period of time. A medically significant period of time is understood by a person of ordinary skill in the art and can indicate, e.g., four cycles or longer, three months or longer. Some or all patients are further administered chemotherapy, the chemotherapeutic agent being gemcitabine, dacarbazine, temozolomide, paclitaxel, albumin-bound paclitaxel, or carboplatin.

Patients are also treated with SEA-CD40 at about 3 μg/kg for multiple cycles of administration, each cycle being 3 weeks, and in combination with pembrolizumab at about 200 mg/kg. SEA-CD40 is administered on one day of each cycle, e.g. day one of each cycle; or on two or more days in each cycle. Pembrolizumab is administered on one day, e.g., day 2 of each cycle; or on two or more days in each cycle. Patients exhibit stable disease without progression for a medically significant period of time. A medically significant period of time is understood by a person of ordinary skill in the art and can indicate, e.g., four cycles or longer, three months or longer. Some or all patients are further administered chemotherapy, the chemotherapeutic agent being gemcitabine, dacarbazine, temozolomide, paclitaxel, albumin-bound paclitaxel, or carboplatin.

More patients are treated with SEA-CD40 at about 10 μg/kg for multiple cycles of administration, each cycle being 3 weeks. SEA-CD40 is administered one day of each cycle, e.g. day one of each cycle; or on two or more days in each cycle. Patients exhibit stable disease without progression for a medically significant period of time. A medically significant period of time is understood by a person of ordinary skill in the art and can indicate, e.g., four cycles or longer, three months or longer. Some or all patients are further administered chemotherapy, the chemotherapeutic agent being gemcitabine, dacarbazine, temozolomide, paclitaxel, albumin-bound paclitaxel, or carboplatin.

Patients are also treated with SEA-CD40 at about 10 μg/kg for multiple cycles of administration, each cycle being 3 weeks, and in combination with pembrolizumab at about 200 mg/kg. SEA-CD40 is administered on one day of each cycle, e.g. day one of each cycle; or on two or more days in each cycle. Pembrolizumab is administered on one day, e.g., day 2 of each cycle; or on two or more days in each cycle. Patients exhibit stable disease without progression for a medically significant period of time. A medically significant period of time is understood by a person of ordinary skill in the art and can indicate, e.g., four cycles or longer, three months or longer. Some or all patients are further administered chemotherapy, the chemotherapeutic agent being gemcitabine, dacarbazine, temozolomide, paclitaxel, albumin-bound paclitaxel, or carboplatin.

More patients are treated with SEA-CD40 at about 30 μg/kg for multiple cycles of administration, each cycle being 3 weeks. SEA-CD40 is administered one day of each cycle, e.g. day one of each cycle; or on two or more days in each cycle. Patients exhibit stable disease without progression for a medically significant period of time. A medically significant period of time is understood by a person of ordinary skill in the art and can indicate, e.g., four cycles or longer, three months or longer. Some or all patients are further administered chemotherapy, the chemotherapeutic agent being gemcitabine, dacarbazine, temozolomide, paclitaxel, albumin-bound paclitaxel, or carboplatin.

Patients are also treated with SEA-CD40 at about 30 μg/kg for multiple cycles of administration, each cycle being 3 weeks, and in combination with pembrolizumab at about 200 mg/kg. SEA-CD40 is administered on one day of each cycle, e.g. day one of each cycle; or on two or more days in each cycle. Pembrolizumab is administered on one day, e.g., day 2 of each cycle; or on two or more days in each cycle. Patients exhibit stable disease without progression for a medically significant period of time. A medically significant period of time is understood by a person of ordinary skill in the art and can indicate, e.g., four cycles or longer, three months or longer. Some or all patients are further administered chemotherapy, the chemotherapeutic agent being gemcitabine, dacarbazine, temozolomide, paclitaxel, albumin-bound paclitaxel, or carboplatin.

More patients are treated with SEA-CD40 at about 45 μg/kg for multiple cycles of administration, each cycle being 3 weeks. SEA-CD40 is administered one day of each cycle, e.g. day one of each cycle; or on two or more days in each cycle. Patients exhibit stable disease without progression for a medically significant period of time. A medically significant period of time is understood by a person of ordinary skill in the art and can indicate, e.g., four cycles or longer, three months or longer. Some or all patients are further administered chemotherapy, the chemotherapeutic agent being gemcitabine, dacarbazine, temozolomide, paclitaxel, albumin-bound paclitaxel, or carboplatin.

Patients are also treated with SEA-CD40 at about 45 μg/kg for multiple cycles of administration, each cycle being 3 weeks, and in combination with pembrolizumab at about 200 mg/kg. SEA-CD40 is administered on one day of each cycle, e.g. day one of each cycle; or on two or more days in each cycle. Pembrolizumab is administered on one day, e.g., day 2 of each cycle; or on two or more days in each cycle. Patients exhibit stable disease without progression for a medically significant period of time. A medically significant period of time is understood by a person of ordinary skill in the art and can indicate, e.g., four cycles or longer, three months or longer. Some or all patients are further administered chemotherapy, the chemotherapeutic agent being gemcitabine, dacarbazine, temozolomide, paclitaxel, albumin-bound paclitaxel, or carboplatin.

More patients are treated with SEA-CD40 at about 60 μg/kg for multiple cycles of administration, each cycle being 3 weeks. SEA-CD40 is administered one day of each cycle, e.g. day one of each cycle; or on two or more days in each cycle. Patients exhibit stable disease without progression for a medically significant period of time. A medically significant period of time is understood by a person of ordinary skill in the art and can indicate, e.g., four cycles or longer, three months or longer. Some or all patients are further administered chemotherapy, the chemotherapeutic agent being gemcitabine, dacarbazine, temozolomide, paclitaxel, albumin-bound paclitaxel, or carboplatin.

Patients are also treated with SEA-CD40 at about 60 μg/kg for multiple cycles of administration, each cycle being 3 weeks, and in combination with pembrolizumab at about 200 mg/kg. SEA-CD40 is administered on one day of each cycle, e.g. day one of each cycle; or on two or more days in each cycle. Pembrolizumab is administered on one day, e.g., day 2 of each cycle; or on two or more days in each cycle. Patients exhibit stable disease without progression for a medically significant period of time. A medically significant period of time is understood by a person of ordinary skill in the art and can indicate, e.g., four cycles or longer, three months or longer. Some or all patients are further administered chemotherapy, the chemotherapeutic agent being gemcitabine, dacarbazine, temozolomide, paclitaxel, albumin-bound paclitaxel, or carboplatin.

### Example 2: T Cell Activation in Uveal Melanoma Patient Treated with SEA-CD40

During the period treated with SEA-CD40 in combination with pembrolizumab, samples from Patient 2 were taken pre-dose of each cycle and analyzed for T cell activation. The results are shown in FIGS. 26A and 26B. High magnitude of T cell activation was observed during treatment and particularly at cycle 4 (C4), coinciding with disease regression on imaging.

SEQUENCE LISTING

```
SEQ ID NO: 1; SEA-CD40 heavy chain
10 20 30 40 50 60
  | | | | | |
EVQLVESGGGLVQPGGSLRLSCAASGYSFTGYYIHWVRQAPGKGLEWVARVIPNAGGTSY
70 80 90 100 110 120
  | | | | | |
NQKFKGRFTLSVDNSKNTAYLQMNSLRAEDTAVYYCAREGIYWWGQGTLVTVSSASTKGP
130 140 150 160 170 180
  | | | | | |
SVFPLAPSSKSTSGGTAALGCLVKDYFPEPVTVSWNSGALTSGVHTFPAVLQSSGLYSLS
190 200 210 220 230 240
  | | | | | |
SVVTVPSSSLGTQTYICNVNHKPSNTKVDKKVEPKSCDKTHTCPPCPAPELLGGPSVFLF
250 260 270 280 290 300
  | | † | | * | |
PPKPKDTLMISRTPEVTCVVVDVSHEDPEVKFNWYVDGVEVHNAKTKPREEQYNSTYRVV
310 320 330 340 350 360
  | | | | | |
SVLTVLHQDWLNGKEYKCKVSNKALPAPIEKTISKAKGQPREPQVYTLPPSREEMTKNQV
370 380 390 400 410 420
  | | | | | |
SLTCLVKGFYPSDIAVEWESNGQPENNYKTTPPVLDSDGSFFLYSKLTVDKSRWQQGNVF
430 440
  | |
SCSVMHEALHNHYTQKSLSLSPGK
SEQ ID NO: 2, SEA-CD40 light chain
10 20 30 40 50 60
  | | | | | |
DIQMTQSPSSLSASVGDRVTITCRSSQSLVHSNGNTFLHWYQQKPGKAPKLLIYTVSNRF
70 80 90 100 110 120
  | | | | | |
SGVPSRFSGSGSGTDFTLTISSLQPEDFATYFCSQTTHVPWTFGQGTKVEIKRTVAAPSV
130 140 150 160 170 180
  | | | | | |
FIFPPSDEQLKSGTASVVCLLNNFYPREAKVQWKVDNALQSGNSQESVTEQDSKDSTYSL
190 200 210
  | | |
SSTLTLSKADYEKHKVYACEVTHQGLSSPVTKSFNRGEC
```

What is claimed is:

1. A method of treating uveal melanoma comprising administering a composition comprising an anti-CD40 antibody to a patient with uveal melanoma,
wherein the anti-CD40 antibody comprises: 1) a heavy chain variable region comprising an amino acid sequence of SEQ ID NO:1; 2) a light chain variable region comprising an amino acid sequence of SEQ ID NO:2; and 3) a human constant region;
wherein the human constant region comprises an N-glycoside-linked sugar chain at residue N297 (EU numbering), wherein less than 5% of N-glycoside-linked sugar chains at residue N297 (EU numbering) in the composition comprise a fucose residue; and
wherein the anti-CD40 antibody is administered at a dose of about 3 μg/kg, about 10 μg/kg, about 30 μg/kg, about 45 μg/kg, or about 60 μg/kg patient body weight.

2. The method of claim 1, wherein the dose is about 30 μg/kg patient body weight.

3. The method of claim 1 or 2, wherein the anti-CD40 antibody is administered every three weeks or every six weeks.

4. The method of any one of claims 1-3, further comprising administering a chemotherapeutic agent.

5. The method of claim 4, wherein the chemotherapeutic agent is gemcitabine, dacarbazine, temozolomide, paclitaxel, albumin-bound paclitaxel, or carboplatin.

6. A method of treating uveal melanoma comprising administering 1) a composition comprising an anti-CD40 antibody; and 2) an anti-PD1 antibody or an anti-PDL1 antibody to a patient with uveal melanoma,
wherein the anti-CD40 antibody comprises: 1) a heavy chain variable region comprising an amino acid sequence of SEQ ID NO:1; 2) a light chain variable region comprising an amino acid sequence of SEQ ID NO:2; and 3) a human constant region;
wherein the human constant region comprises an N-glycoside-linked sugar chain at residue N297 (EU numbering), wherein less than 5% of the N-glycoside-linked sugar chains at residue N297 (EU numbering) in the composition comprise a fucose residue; and
wherein the anti-CD40 antibody is administered at a dose of about 3 μg/kg, about 10 μg/kg, about 30 μg/kg, about 45 μg/kg, or about 60 μg/kg patient body weight.

7. The method of claim 6, wherein the anti-PD1 antibody is selected from the group consisting of Pembrolizumab, Nivolumab, Cemiplimab-rwlc, Spartalizumab, AK105, Tislelizumab, Dostarlimab, MEDI0680, Pidilizumab, AMP-224, and SHR-1210.

8. The method of claim 6 or 7, wherein the anti-PDL1 antibody is selected from the group consisting of Atezolizumab, Durvalumab, Avelumab, SHR-1316, MEDI4736, BMS-936559/MDX-1105, MSB0010718C, MPDL3280A, or Envafolimab.

9. The method of any one of claims 6 to 8, wherein the dose is about 30 μg/kg patient body weight.

10. The method of any one of claims 6-9, wherein the anti-CD40 antibody is administered every three weeks or every six weeks.

11. The method of any one of claims 6-10, further comprising administering a chemotherapeutic agent.

12. The method of claim 11, wherein the chemotherapeutic agent is gemcitabine, dacarbazine, temozolomide, paclitaxel, albumin-bound paclitaxel, carboplatin, or paclitaxel.

13. The method of any one of claims 6-12 comprising administering 1) an anti-CD40 antibody and 2) an anti-PD1 antibody.

14. The method of claim 13, wherein the anti-PD1 antibody is pembrolizumab.

15. The method of claim 13 or 14, wherein the anti-PD1 antibody is administered at about 200 mg/kg.
